SEQUENCE LISTING (3 sequences)

SEQ ID NO: 1 - DNA, 24 bp, synthetic construct
  source 1..24: mol_type = other DNA
ggggccggggccggggccggggcc

SEQ ID NO: 2 - RNA, 10 nt, synthetic construct
  source 1..10: mol_type = other RNA
ggggccgggg

SEQ ID NO: 3 - DNA, 174 bp, synthetic construct
  source 1..174: mol_type = other DNA
ggggccggggccggggccggggccggggccggggccggggccggggccggggccggggccggggccggggccggggccggggccggggccggggccggggccggggccggggccggggccggggccggggccggggccggggccggggccggggccggggccggggccggggcc

(19) United States
(12) Patent Application Publication
(10) Pub. No.: US 2026/0199329 A1
(43) Pub. Date: Jul. 16, 2026

(54) SMALL MOLECULES FOR THE RESCUE OF AMYOTROPHIC LATERAL SCLEROSIS (ALS) AND FRONTOTEMPORAL DEGENERATION (FTD) BY TARGETING C9ORF72 HEXANUCLEOTIDE G-QUADRUPEXES

(71) Applicant: The Hong Kong University of Science and Technology, Hong Kong (CN)

(72) Inventors: Guang ZHU, Hong Kong (CN); Changdong LIU, Hong Kong (CN); Ching Monica SUEN, Hong Kong (CN)

(21) Appl. No.: 19/448,352

(22) Filed: Jan. 14, 2026

Related U.S. Application Data
(60) Provisional application No. 63/744,866, filed on Jan. 14, 2025.

(51) Int. Cl. A61K 31/4985; A61K 31/4745; A61P 25/28

(52) U.S. Cl. CPC A61K 31/4985; A61K 31/4745; A61P 25/28

(57) ABSTRACT

The subject invention pertains to a composition comprising a series of compounds selectively targeting G-quadruplexes (G4s) formed by the GGGGCC (G4C2) hexanucleotide repeats (HREs) of C9orf72, (G4C2)ₙ. The composition comprises compounds G4008, G4009, G4010, G4012, and G4013, or a pharmaceutically acceptable salt, solvate, or hydrate thereof and at least one pharmaceutically acceptable carrier and/or excipient. The subject invention further pertains to a method for treating amyotrophic lateral sclerosis (ASL) and/or frontotemporal dementia (FTD), comprising administering to the subject an effective amount of the composition. The compounds can ameliorate the pathological signatures of ALS model caused by C9orf72 (G4C2)ₙ sequence.

### CROSS-REFERENCE TO RELATED APPLICATION

This application claims the benefit of U.S. Provisional Application Ser. No. 63/744,866, filed Jan. 14, 2025, which is hereby incorporated by reference in its entirety including any tables, figures, or drawings.

### SEQUENCE LISTING

The Sequence Listing for this application is labeled “HKUS-206X-SeqList.xml” which was created on Dec. 12, 2025 and is 3,798 bytes. The entire contents of the sequence listing is incorporated herein by reference in its entirety.

### BACKGROUND OF THE INVENTION

Amyotrophic lateral sclerosis (ALS), also referred to as Lou Gehrig's disease, is a devastating neurodegenerative disease characterized by a rapidly progressive loss of motor neurons, ultimately resulting in respiratory failure within 3-5 years after the onset of symptoms [1]. In particular, about 50% of ALS patients present cognitive impairments and 13% of patients develop concomitant frontotemporal dementia (FTD) [2]. ALS affects approximately two individuals per 100,000 characterized by the degeneration of motor neurons, resulting in muscle weakness and atrophy [3].

Regarding the etiology of ALS, it has been reported that sporadic ALS (sALS) constitutes approximately 90% of all ALS cases, while the remaining 10% of ALS patients have familial ALS (fALS), which is caused by genetic mutations [4]. The dysfunction of SOD1, FUS/TLS, and TDP-43 caused by mutation leads to neurotoxicity which results in fALS [5]. Moreover, it has been shown that the expansion of the GGGGCC (G4C2) hexanucleotide repeat sequence in the non-coding region of the C9orf72 gene is a causal factor in both ALS and FTD [6,7]. The abnormal expansion of the G4C2 repeats is observed in 8% of patients with sporadic ALS (sALS) and in over 40% of familial ALS (fALS) cases [8]. Individuals with ALS typically have an average repeat count ranging from 700 to 1600, whereas healthy individuals possess fewer than 25 repeats [9]. In particular, the abnormal expansion of short nucleotide repeats has been identified in numerous neurodegenerative diseases such as (CAG)ₙ leading to Spinocerebellar Ataxia 3 (SCA3) [10]. Therefore, elucidating the pathological mechanisms of these abnormal nucleotide repeats leading to fatal diseases has been a central focus of research, with the goal of discovering effective treatments.

Currently, there are three proposed mechanisms to elucidate the pathological basis of the abnormal expansion of hexanucleotide repeats, (G4C2)ₙ [11]. First, the aberrant G4C2 expansions can lead to a gain or loss of function of C9orf72 gene. Second, the transcribed RNA G4C2 expansions can form toxic RNA foci through recruiting RNA binding proteins (RBPs), resulting in the impairment of the protein function. Third, the non-AUG translation of the RNA G4C2 expansions can produce neurotoxic dipeptide repeats (DPRs) in the central nervous system, leading to the death of neurons. Notably, the mechanism of the formation of toxic RNA foci has received the most attention. In particular, the C9orf72 G4C2 repeats, both DNA and RNA sequence, can fold into secondary structures such as G4 and hairpin [12-15]. Most importantly, the G4 formation is a significant aspect of the toxicity of C9orf72 G4C2 repeats in ALS/FTD, and it was reported that the RNA foci formed by C9orf72 G4C2 repeats are mainly composed of RNA G4s [4, 16]. Consequently, the structural investigation of C9orf72 G4C2 repeats is currently a highly prominent area of research, and the corresponding structures could be the drug targets for the potential treatment.

At present, ALS/FTD is still an incurable disease due to the poor understanding of its disease mechanisms. The United States Food and Drug Administration (FDA) has currently approved seven drugs for the treatment of ALS: Qalsody®, Relyvrio®, Radicava®, Rilutek®, Tiglutik®, Exservan®, and Nuedexta® [17-19]. Qalsody® or tofersen, approved in 2023, is an antisense oligonucleotide targeting mRNA for the mutated superoxide dismutase 1 (SOD1) gene [20]. Relyvrio® is a fixed combination of sodium phenylbutyrate and taurursodiol that prevents nerve cell death by inhibiting stress signals of mitochondria and endoplasmic reticulum in cell, which was approved in 2022 [21] but has been discontinued and removed from the market in the U.S. and Canada in 2024 based on the results of the phase 3 trial. Radicava® or edaravone was firstly marketed and manufactured in Japan in 2015 and approved by FDA in 2017 for an ALS treatment, and is a potent free radical scavenger against reactive oxygen species-driven motor neuron death and inflammation [22]. Rilutek® or riluzole is the first FDA approved ALS drug in 1995, and is an inhibitor of the sodium channel alpha subunit, shown to prolong ALS patients' lives by about three months [23]. Tiglutik® revived FDA approval in 2018, which is an oral suspension variant of riluzole designed for ALS patients who have difficulty in swallowing tablets [24]. Exservan® is an oral film of riluzole specifically designed for ALS patients experiencing severe swallowing difficulties, which was approved in 2019 [25]. Nuedexta® approved in 2010, is an orally administered combination of dextromethorphan hydrobromide and quinidine sulfate for the treatment of excessive emotional lability, known as pseudobulbar affect (PBA) [26]. However, the detailed mechanisms of these drugs in treatment of ALS remain elusive. Particularly, each year more than 5000 adding cases are diagnosed as ALS in the world. Therefore, there is an urgent clinical demand for discovering and identifying effective novel targets and drugs for the treatment of the ALS.

### BRIEF SUMMARY OF THE INVENTION

In one aspect, the subject invention pertains to a novel composition for treating ALS, comprising derivatives of 7H-Pyridocarbazole and 7H-pyrazinocarbazole, selectively targeting G-quadruplexes (G4s) formed by the GGGGCC (G4C2) hexanucleotide repeats (HREs) of C9orf72, (G4C2)ₙ. In certain embodiments, the composition comprises one or more compound comprising G4008, G4009, G4010, G4012, and G4013, or a pharmaceutically acceptable salt, solvate, or hydrate thereof, and at least one pharmaceutically acceptable carrier and/or excipient.

In another aspect, the subject invention pertains to a novel method for treating ALS comprising administering to a subject an effective amount of the composition of the subject invention. In certain embodiments, the novel compounds of the subject invention target C9orf72 G4C2 G4s. In preferred embodiments, the composition comprises one or more compound selected from the group consisting of G4008, G4010, G4012, and G4013, or a pharmaceutically acceptable salt, solvate, or hydrate thereof and at least one pharmaceutically acceptable carrier and/or excipient. In more preferred embodiments, the composition comprises one or more compound selected from the group consisting of G4010 and G4013, or a pharmaceutically acceptable salt, solvate, or hydrate thereof and at least one pharmaceutically acceptable carrier and/or excipient. In most preferred embodiments, the composition comprises compound G4013, or a pharmaceutically acceptable salt, solvate, or hydrate thereof and at least one pharmaceutically acceptable carrier and/or excipient.

In certain embodiments, the administration of the composition comprising one or more compound selected from the group consisting of G4008, G4010, G4012, and G4013 significantly reduces RNA foci number and reactive oxygen species (ROS) level in the subject.

In certain embodiments, the administration of the composition can significantly ameliorate the pathological signatures of ALS model caused by C9orf72 (G4C2)ₙ sequence. In further embodiments, the administration of the composition can significantly ameliorate one or more symptoms and/or the progression of ALS and/or FTD in a subject.

### BRIEF DESCRIPTION OF THE DRAWINGS

FIG. 1 illustrates the structure of 6 compounds designed based on chrexanthomycin A. FIG. 1 shows the designed small molecules G4008-G4013 based on chemical structural scaffold of chrexanthomycin A, derivatives of 7H-Pyridocarbazole and 7H-pyrazinocarbazole.

FIG. 2 shows the NMR titration of the compounds with C9orf72 DNA (G4C2)₄ G4. The imino region of 1D 1H spectra of C9orf72 DNA (G4C2)₄ titrated with compounds (free: black, 1:1 red, 1:5 green, 1:10 purple) are illustrated for small molecules G4008, G4009, G4010, G4011, G4012, G4013.

FIG. 3 illustrates the NMR titration of the compounds with C9orf72 RNA (G4C2G4) G4. The imino region of 1D ¹H spectra of C9orf72 DNA (G4C2)₄ titrated with compounds (free: black, 1:1 red, 1:5 green, 1:10 purple) are illustrated for small molecules G4008, G4009, G4010, G4011, G4012, G4013.

FIGS. 4A-4B illustrate the cellular tests of the compounds in HT22 cells by MTT and ROS assay. FIG. 4A illustrates MTT assay of 6 compounds (six data points correspond to 0.064 μM, 0.32 μM, 1.6 μM, 8 μM, 40 μM, and 200 μM presented in log concentration). FIG. 4B illustrates ROS assay of 6 compounds (x axis: 6 compounds and relevant control group, y axis: normalized ROS fluorescence intensity).

FIGS. 5A-5B illustrate the compounds rescue C9orf72 (G4C2)₂₉ expansion-related pathology in cell model. FIG. 5A shows a representative RNA FISH images of Neuro2a cells transfected with C9orf72 DNA (G4C2)₂₉ in DMSO control or new compounds (8 μM treatment). RNA foci of C9orf72 G4C2 repeats was detected by Cy3 labelled probe while DAPI was applied as counterstain (blue). FIG. 5B illustrates the quantification of C9orf72 RNA G4C2 foci numbers per cell in (G4C2)₂₉ expressed cells treated with new compounds. n=15-20 cells, p values were determined by one-way ANOVA.

FIGS. 6A-6B illustrate the compounds rescue C9orf72 (G4C2)₄₉ expansion-related pathology in Drosophila model. FIG. 6A shows representative external eye images and scanning electron microscopy (SEM) images of 7-day-old wild-type (WT) and GMR-GAL4-(G4C2)₄₉ Drosophila fed with DMSO (100 μM), compounds G4010 (100 μM) and G4013 (100 μM) during the larval stage. FIG. 6B illustrates the quantification of the eye degeneration percentage of WT and GMR-GAL4-(G4C2)₄₉ Drosophila fed with DMSO (100 μM), compounds G4010 (100 μM), and G4013 (100 μM). n=7-18 animals; p values were determined by one-way ANOVA (Tukey's multiple comparisons test).

FIG. 7 illustrates the ITC experiments of G4010 and G4013 with C9orf72 DNA (G4C2)₄ and RNA (G4CG4) G4. The ITC experiments were performed in a buffer containing mM potassium phosphate (pH7.0), 70 mM KCl for G4010+DNA (G4C2)₄, G4010+RNA (G4C2G4), G4013+DNA (G4C2)₄, and G4013+RNA (G4C2G4). The compounds were dissolved in DMSO with stock solution at the concentration of 10 mM.

### BRIEF DESCRIPTION OF THE SEQUENCES

SEQ ID NO: 1 The DNA sequence d[GGGGCCGGGGCCGGGGCCGGGGCC], corresponding to the C9orf72 DNA (G4C2)₄, consists of 24 nucleotides and was purchased by Accurate Biotechnology (Hunan) Co., Ltd.

SEQ ID NO: 2 The RNA sequence r[GGGGCCGGGG], corresponding to the C9orf72 RNA (G4C2G4), consists of 10 nucleotides and was synthesized by Accurate Biotechnology (Hunan) Co., Ltd. Tomorrow

SEQ ID NO: 3 The DNA sequence d[GGGGCCGGGGCCGGGGCCGGGGCCGGGGCCGGGGCCGGGGCCGGGGCCGGGGCC GGGGCCGGGGCCGGGGCCGGGGCCGGGGCCGGGGCCGGGGCCGGGGCCGGGGCCG GGGCCGGGGCCGGGGCCGGGGCCGGGGCCGGGGCCGGGGCCGGGGCCGGGGCCGG GGCCGGGGCC], corresponding to the C9orf72 DNA (G4C)₂₉, consists of 174 nucleotides which was inserted into the DNA plasmid pHR-Tre3G-29xGGGGCC-12xMS2 (Addgene #99149) purchased form Addgegne.

### DETAILED DISCLOSURE OF THE INVENTION

### Selected Definitions

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof, are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting”, and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” are used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, particularly a human, needing or desiring delivery of the benefits provided by a therapeutic compound. The term “subject” as used herein, encompasses both human and non-human animals. The term “non-human animals” includes all vertebrates, e.g., mammals, such as non-human primates, (particularly higher primates), sheep, dog, rodent (e.g., mouse or rat), guinea pig, goat, pig, cat, rabbits, cows, and non-mammals, such as chickens, amphibians, reptiles etc. These benefits can include, but are not limited to, the treatment of a health condition, disease, or disorder; prevention of a health condition, disease or disorder; immune health; enhancement of the function of an organ, tissue, or system in the body. In one embodiment, the subject is human. In another embodiment, the subject is an experimental animal or animal substitute as a disease model. In some embodiments, the term “subject” refers to a mammal, including, but not limited to, murines, simians, humans, felines, canines, equines, bovines, mammalian farm animals, mammalian sport animals, and mammalian pets. The preferred subject in the context of this invention is a human. The term does not denote a particular gender. Thus, male and female subjects are intended to be covered. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, inhibiting, or improving a condition, disease, or disorder in a subject. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the term “treatment” refers to eradicating; reducing; inhibiting; ameliorating; abatement; remission; diminishing of symptoms, or delaying the onset of symptoms; slowing in the rate of degeneration or decline; making the final point of degeneration less debilitating; and/or improving a subject's physical or mental well-being or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, the terms “arresting”, “reducing”, “inhibiting”, “blocking”, “preventing”, “alleviating”, “delaying”, “forestalling”, “minimizing”, or “relieving” the onset of a particular sign or symptom of the condition, disease, or disorder. Inhibition can, but is not required, to be absolute or complete; meaning, the sign or symptom may still develop at a later time. Inhibition can include reducing the severity of the onset of such a condition, disease, or disorder, and/or inhibiting the progression of the condition, disease, or disorder to a more severe condition, disease, or disorder.

When referring to a compound, “inhibition” means that the compound brings down the occurrence, severity, size, volume or associated symptoms of ALS by at least about 20%, 25%, 30%, 35%, 40%, 45%, 50%, 55%, 60%, 65%, 70%, 75%, 80%, 90%, or 100% compared to how the pathological signatures of ALS/FTD model would normally exist without application of the compounds or compositions comprising the compound.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences, as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with gene, cDNA, and mRNA encoded by a gene.

As used herein, the terms “oligos”, “oligonucleotides” are used interchangeably to describe short single strands of synthetic DNA or RNA, such as, for example, about a 5 nucleic acid base sequence to about a 500 nucleic acid base sequence. “Oligo delivery” and “oligonucleotide delivery” are also used interchangeably to describe the method by which oligonucleotides are delivered into cells or targeted to specific cell types, tissues, or organs.

The term “G-quadruplex” refers to its ordinary and customary meaning. A “G-quadruplex” is a square structural arrangement of four guanines that is stabilized by Hoogsteen hydrogen bonds. A “G-quadruplex” is helical in shape and formed from interconnected stacks of guanine tetrads (or “G-tetrads”), which individually are flat, ring-shaped structures formed from four guanines, and can also be stabilized by the presence of a monovalent cation (e.g., potassium) in the center of the tetrad. A “G-quadruplex” can be formed by a DNA, an RNA, or any combination thereof.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

In certain embodiments, treatments are administered to a subject that significantly reduce or abolish RNA foci formation and foci number in neurons. This phrase refers to a reduction of foci number in an amount of at least (or at least about) 30% as compared to a non-treated subject. Thus, the treated subjects exhibit significantly reduced or abolished RNA foci number that can range from about 30% to about 99.99%, about 35% to about 99.99%, about 40% to about 99.99%, about 45% to about 99.99%, about 50% to about 99.99%, about 60% to about 99.99%, about 70% to about 99.99%.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

“Pharmaceutically acceptable salt” refers to a salt of a compound of the invention that is pharmaceutically acceptable and that possesses the desired pharmacological activity of the parent compound. In particular, such salts that are non-toxic may be inorganic or organic acid addition salts and base addition salts.

“Pharmaceutically acceptable vehicle” refers to a diluent, adjuvant, excipient or carrier with which a compound of the invention is administered. A “pharmaceutically acceptable vehicle” refers to a substance that is non-toxic, biologically tolerable, and otherwise biologically suitable for administration to a subject, such as an inert substance, added to a pharmacological composition or otherwise used to facilitate administration of an agent and that is compatible therewith. Examples of vehicles include but are not limited to calcium carbonate, calcium phosphate, various sugars and types of starch, cellulose derivatives, gelatin, vegetable oils, and polyethylene glycols.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, the terms “determining,” “measuring,” and “assessing,” and “assaying” are used interchangeably and include both quantitative and qualitative determinations.

In some embodiments of the invention, the method comprises administration of multiple doses of the compositions of the subject invention. The method may comprise administration of therapeutically effective doses of a composition comprising the compound or composition thereof of the subject invention as described herein twice a day, once a day, every other day, three times a week, once a week, once a month, or a lower frequency. In some embodiments, doses are administered over the course of 1 week, 2 weeks, 3 weeks, 4 weeks, 2 months, 6 months, 1 year, 2 years, 3 years, 4 years, 5 years, 6 years, 7 years, 8 years, 9 years, 10 years, or more than 10 years. Moreover, treatment of a subject with a therapeutically effective amount of the compositions of the invention can include a single treatment or can include a series of treatments. It will also be appreciated that the effective dosage of a compound or composition thereof used for treatment may increase or decrease over the course of a particular treatment. Changes in dosage may result and become apparent from the results of diagnostic assays, which are known in the art. In some embodiments of the invention, the method comprises administration of the compositions from once a day to several time per day, including but not limiting to 2 times per day, 3 times per day, and 4 times per day.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Methods of the Subject Invention

In one aspect, the subject invention pertains to a novel composition for treating amyotrophic lateral sclerosis (ASL) and/or frontotemporal dementia (FTD), comprising one or more compound including, but not limited to, G4008, G4009, G4010, G4012, and G4013, or a pharmaceutically acceptable salt, solvate, or hydrate thereof and at least one pharmaceutically acceptable carrier and/or excipient.

In another aspect, the subject invention pertains to a method for treating ALS and/or FTD comprising administering to a subject an effective amount of a composition comprising one or more compounds including, but not limited to, G4008, G4009, G4010, G4012, and G4013. In certain embodiments, the one or more compounds of the composition can selectively recognize C9orf72 G4C2 G4s in a cell. In preferred embodiments, the administration of the composition to a subject significantly reduces, inhibits, or ameliorates one or more symptoms of ASL and/or FTD by about 70%. In preferred embodiments, significantly reduces, inhibits, or ameliorates the progression of ASL and/or FTD by about 70%.

In preferred embodiments, the composition includes, but is not limited to, compounds G4008, G4010, G4012, and G4013, or a pharmaceutically acceptable salt, solvate, or hydrate thereof and at least one pharmaceutically acceptable carrier and/or excipient. In more preferred embodiments, the composition includes, but is not limited to, compounds G4010 and G4013, or a pharmaceutically acceptable salt, solvate, or hydrate thereof and at least one pharmaceutically acceptable carrier and/or excipient. In most preferred embodiments, the composition comprises compound G4013, or a pharmaceutically acceptable salt, solvate, or hydrate thereof and at least one pharmaceutically acceptable carrier and/or excipient. In certain embodiments, the composition comprises compound G4013 as the only active agent, or a pharmaceutically acceptable salt, solvate, or hydrate thereof and at least one pharmaceutically acceptable carrier and/or excipient. In certain embodiments, the composition includes, but is not limited to, compound G4013, or a pharmaceutically acceptable salt, solvate, or hydrate thereof and at least one pharmaceutically acceptable carrier and/or excipient.

In certain embodiments, RNA foci number in cells a subject affected by ALS and/or FTD are significantly reduced by the administration of the composition comprising one or more of compounds G4008, G4010, G4012, and G4013. In certain embodiments, the number of foci is reduced by at least 20%, 25%, 30%, 35%, 40%, 45%, 50%, or more. In preferred embodiments, the number of foci is reduced by an average of about 50%, or more.

In certain embodiments, the administration of the composition significantly reduces reactive oxygen species (ROS) level of cells in the subject.

In preferred embodiments, the administration of G4013 targets C9orf72 G4C2 G4s with a high binding affinity, where the binding affinity (K_{d}) of G4013 with C9orf72 DNA (G4C2)₄ G4s is about 114 μM, and with C9orf72 RNA (G4C2G4) G4s is about 68 μM.

In certain embodiments, the subject is a mammal. In preferred embodiments, the mammal is a primate. In more preferred embodiments, the primate is a human. In certain embodiments, the subject is affected by ALS and/or FTD.

In certain embodiments, the therapeutically effective amount of the compositions of the instant invention may be formulated for parenteral administration e.g., by injection, for example, bolus injection, intravenous administration, intraperitoneal administration, or continuous infusion, oral, rectal, bronchial, nasal, topical, buccal, sub-lingual, transdermal, vaginal, intramuscular, intra-arterial, intracerebral, intraocular administration or in a form suitable for administration by inhalation or insufflation, including powders and liquid aerosol administration, or by sustained release systems such as semipermeable matrices of solid hydrophobic polymers. Administration may be also by way of other carriers or vehicles such as patches, micelles, liposomes, vesicles, implants (e.g. microimplants), synthetic polymers, microspheres, nanoparticles, and the like.

The compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

In various embodiments the active agents described herein (e.g., G4008, G4009, G4010, G4012, and G4013) can be combined with a pharmaceutically acceptable carrier (excipient) to form a pharmacological composition. In certain embodiments, pharmaceutically acceptable carriers include those approved by a regulatory agency of the Federal or a state government or listed in the U.S. Pharmacopeia or other generally recognized pharmacopeia for use in/on animals, and more particularly in/on humans. A “carrier” refers to, for example, a diluent, adjuvant, excipient, auxiliary agent or vehicle with which an active agent of the present invention is administered.

In addition, the compositions may be presented in unit dose form in ampoules, pre-filled syringes, and small volume infusion or in multi-dose containers with or without an added preservative. The compositions may be in forms of suspensions, solutions, or emulsions in oily or aqueous vehicles. The composition may further contain formulation agents such as suspending, stabilizing and/or dispersing agents. In further embodiments, the active ingredients of the compositions according to the instant invention may be in powder form, obtained by aseptic isolation of sterile solid or by lyophilization from solution for constitution with a suitable vehicle, e.g., sterile, pyrogen-free water, before use.

The subject compositions can further comprise one or more pharmaceutically acceptable carriers, and/or excipients, and can be formulated into liquid forms, such as solutions for injections or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g., carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds include wetting agents, emulsifying agents, dispersing agents or preservatives that are particularly useful for inhibiting the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In one embodiment, the excipients are sterile and generally free of undesirable matter. These compositions can be sterilized by conventional, well-known sterilization techniques.

In certain therapeutic applications, the compound or composition are administered therapeutically to a subject planned for receiving a chemotherapy treatment in an amount sufficient to reduce, and/or inhibit the symptoms, morbidity, severity, and/or progression of the disease. An amount adequate to accomplish this is defined as a “therapeutically effective dose.” Amounts effective for therapeutic use will depend upon the expected response of the subject to the active agents. Single or multiple administrations of the active agents may be utilized depending on the dosage and frequency as required and tolerated by the subject. In any event, the treatment should provide a sufficient amount of the compound or composition to effectively reduce, inhibit, or ameliorate one or more symptoms and the progression of ASL and/or FTD.

The concentration/amount of the compound or composition can vary widely, and are selected primarily based on activity of the active ingredient(s), body weight and the like in accordance with the particular mode of administration selected and the patient's needs. Concentrations, however, will typically be selected to provide dosages ranging from about 0.0005 mg/kg/day to about 1,000 mg/kg/day and sometimes higher. Typical dosages range from about 1.0 mg/kg/day to about 250 mg/kg/day, preferably from about 1.0 mg/kg/day to about 200 mg/kg/day, more preferably from about 1.0 mg/kg/day to about 150 mg/kg/day, and most preferably from about 1.0 mg/kg/day to about 100 mg/kg/day. the dosages may range preferably from about 600 mg/kg/day to about 1,000 mg/kg/day, more preferably from 600 mg/kg/day to about 750 mg/kg/day; and most preferably from 600 mg/kg/day to about 650 mg/kg/day. It will be appreciated that such dosages may be varied to optimize a therapeutic regimen in a particular subject.

In certain examples, the composition is administered in an amount of about 10 mg/kg to about 250 mg/kg of the subject body weight. In preferred examples, the composition is administered in an amount of about 15 mg/kg to about 200 mg/kg of the subject body weight. In more preferred examples, the composition is administered in an amount of about 25 mg/kg to about 200 mg/kg of the subject body weight.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, or more than 30 days, or once a week, once a month, once a quarter, twice a year, once a year, or a lower frequency. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic assays or imaging techniques for detecting the progression ALS or FTD known in the art. In some embodiments of the invention, the method comprises administration of the compounds at several time per day, including but not limiting to 2 times per day, 3 times per day, and 4 times per day. In some embodiments, the compound or composition is administered to a patient for about three to about five days.

In certain embodiments, the administration of at least one dose of the composition is repeated at least daily, biweekly, thrice-weekly, weekly, bimonthly, monthly, yearly, or at least every about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 52 weeks, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, or longer. In certain embodiments, the repeated administrations of at least one of dose of the composition occurs for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 52 weeks, or longer. In preferred embodiments, the administration of at least one dose of the composition is repeated every about 2 days to about 5 days for about 1 year, about 2 years, about 3 years, about 4 years, about 5 years, about 6 years, about 7 years, about 8 years, about 9 years, to about 10 years. In preferred embodiments, the composition is administered once daily for about 8 weeks to about 52 weeks. In more preferred embodiments, the administration of at least one dose of the composition is repeated every about 2 days to about 5 days for about 1 years to about 5 years. Further embodiments of the present invention including but not limited to the following experimental methods and results:

### Materials and Methods

### G4 DNA/RNA Sample Preparation

The DNA/RNA oligonucleotides oligos were purchased from Accurate Biotechnology (HUNAN) Co., LTD. 100 μM concentration of DNA/RNA samples were annealed at 97° C. in annealing buffer containing 20 mM potassium phosphate (pH7.0), 70 mM KCl, followed by slow cooling overnight to room temperature. For 1D NMR screening, samples were prepared in 50 μM concentration with 5% D2O.

### NMR Spectroscopy

Experiments were performed on 500 MHz and 800 MHz Varian spectrometers. All samples were diluted to 50 μM from initial stock and made up to 400 μL final volume for NMR test. Guanine imino protons were identified using ¹H NMR spectra and data were analyzed by MestReNova. NMR titrations were carried out by adding compounds which are pre-dissolved in DMSO-d6 with stock concentration as 20 mM.

### One-Dimensional ¹H-NMR Titration Experiments

To verify whether or not the compounds interact with G4 DNA/RNA, we performed NMR titration experiments by running one-dimensional (1D) ¹H NMR spectra at 25° C. All compounds were dissolved in isotope labeled d₆-DMSO at about 100 mM concentration, working as stock solutions. To avoid chemical shift changes of G4 DNA/RNA resulted from d₆-DMSO addition, 10 or 15 μL d₆-DMSO were added into 400 μL of 0.1 mM G4 DNA/RNA solution in the NMR buffer (20 mM potassium phosphate buffer and 70 mM KCl, pH 7.0, 10% D₂O), respectively, and then 1D 1H-NMR spectra were conducted. During NMR titration experiments of each compound into G4 DNA/RNA solution, the maximal volume 10 or 15 μL of each compound in d₆-DMSO solution was thought as a final data point.

### Isothermal Titration Calorimetry (ITC)

ITC is commonly used for quantitative analysis of an interaction between two molecules. It provides valuable information such as binding stoichiometry, kinetics, and affinities of the complex formation via the association and dissociation constant. Malvern Panalytical MicroCal PEAQ-ITC was applied for measuring the affinities between ligands and DNA/RNA. The sample preparation is required to be at least 400 μL since the cell volume is 280 μL whereas 40 μL of ligand is needed as titrant. Mole ratio is subjected to case, usually begins with 10-20:1 (titrant: cell). All samples have to be maintained in the same buffer conditions including additives. 18 injections is normally used as default titration program with 2 μL of titration for each injection (0.4 μL for the first injection).

### Cell Culture

Neuro2a cells were seeded on glass coverslips using a 12-well plate initially. DNA plasmids pHR-Tre3G-29xGGGGCC-12xMS2 (Addgene #99149) and pCAG-TetON-3G plasmid (Addgene #96963) were then co-transfected to cells using Lipofectamine 3000 (ThermoFisher Scientific). Each well was transfected with 1 μg of plasmid according to the manufacturer's protocol. Plasmids were incubated over 10 hours for expression and induced by doxycycline (1000 ng/mL) for overnight at 37° C. in incubator.

### Transfection

DNA constructs were transfected at 7 DIV using Lipofectamine 2000 (Thermo Fisher Scientific, 11668019) or Lipofectamine LTX with Plus Reagent (Thermo Fisher Scientific, 15338100). 4 to 6 h after transfection, fresh culture medium was used to replace the transfection medium, and the cells were incubated for another 48 h (HT22) or longer (primary neurons; 5-8 d) to allow recovery and construct expression.

### MTT Assay

The use of MTT assay ((3-[4,5-dimethylthiazol-2-yl]-2,5 diphenyl tetrazolium bromide) is to measure the cytotoxicity levels of compounds at different concentrations to determine cell growth inhibition (IC50) [27]. Mitochondrial activity of cells could be reflected by converting the tetrazolium salts into formazan crystals, which is further dissolved by DMSO for measurements. 5×10³ number of HT22 cells were first seeded in 96-well plate and cultured overnight. The cells would then be treated with various concentrations of compounds added and diluted in medium for overnight. 10 μL of MTT (5 mg/ml) diluted in PBS buffer was added into each well and incubated for 4 hours at 37° C. 100 μL of DMSO was then added to each well to dissolve the crystal which was prewashed thoroughly. Absorbance was recorded afterwards with a CLARIOstar PLUS plate reader (BMG LABTECH) using 570 nm wavelength. IC50 was then analyzed by GraphPad Prism.

### Reactive Oxygen Species ROS Assay

HT22 cells were prepared in 96 well plate and DMEM medium supplemented with 5 mM L-Glutamate acid and incubated overnight at 37° C. Then, ROS assay kit was applied for testing the reactive oxygen level according to the manufacturer's protocol (Beyotime). 10 μM DCFH-DA fluorescent probe was added to the cells, which were prewashed with DMEM only medium for a 30 minutes incubation at 37° C. with gentle shaking every 5 minutes. Cells were then washed with DMEM-only medium to remove excess probe for three times. Respective compounds (2 μM) and controls (Rosup: 50 μg/mL) were added afterwards and incubated for an hour. Fluorescence intensity of the oxidated products dichlorofluorescein (DCF) could be measured with a CLARIOstar PLUS plate reader (BMG LABTECH) using 488/525 nm filters.

### Drosophila Strains

(G4C2)ₙ repeats relevant fly lines and eye development expression promoter were obtained from Bloomington Drosophila Stock Centre (BDSC) while wild type strains e.g., w(CS10) were gifted from Prof. Yukinori's group. All Drosophilae were raised at 25° C. with humidity controlled between 56-60%. Fly food was prepared one day before adding with DMSO control and respective promising compounds at 100 μM. Parent flies were then crossed next day on solidified food containing the testing compounds and larvae were also fed with food in compounds until eclosion.

### Eye Imaging Using Bright Field Microscope

Eye images of all adult flies after eclosion were anesthetized with CO₂ and taken on day 5 using camera (Canon EOS Kiss X7 DOUBLE ZOOM KIT) on microscope (Nikon SMZ 745T) to capture the whole eye situation before proceeding to scanning electron microscope. Eye degeneration level would then be examined accordingly and collected separately.

### Eye Imaging Using Scanning Electron Microscopy (SEM)

Flies which were separated during the previous section were further treated with 2.5% glutaraldehyde for fixation at 4° C. overnight. Then, flies were dried in graded acetone for 12 hours accordingly per day (30%, 50%, 70%, 90%, 95%, 100% twice). Dried flies were then air dried and compound eyes were dissected using light microscope. Finally, gold coating of compound eyes would be carried out before SEM (JSM-6390).

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Design of Novel Compounds Based on Chrexanthomycin A

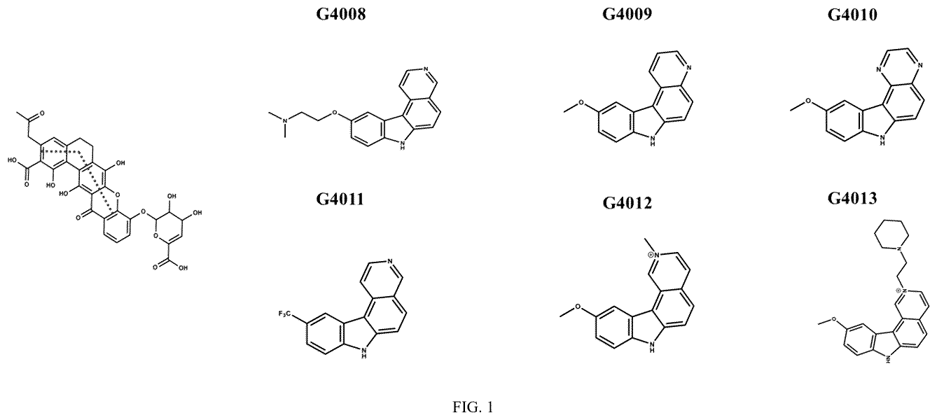

ALS is a fatal degenerative neurological disease that currently have no cure. The abnormal expansion of a hexanucleotide, (G4C2)ₙ (n from 30 up to thousand), in the first non-coding region of C9orf72 gene is the most common genetic cause of ALS/FTD. The C9orf72 DNA/RNA G4C2 repeats can fold into a secondary structure, G4. Several studies revealed that C9orf72 G4C2 G4s potentially are novel drug targets by small molecules in treatment of ALS [28-30]. In our previous study, we identified a marine-derived natural product, chrexanthomycin A, which can specifically recognize C9orf72 DNA/RNA G4C2 G4. However, the binding affinity of chrexanthomycin A is weak with K_{d}~2.2±0.1, 3.0±0.1, and 2.8±0.1 mM. To develop leading compounds targeting C9orf72 DNA/RNA G4C2 G4 with high selectivity, specificity and affinity, we designed and synthesized 6 small molecules (G4008-G4013), which are derivatives of 7H-Pyridocarbazole and 7H-pyrazinocarbazole, based on the structure of chrexanthomycin A, and containing 4 fused rings. (FIG. 1).

### Example 2—The NMR Titration of these Designed Compounds with C9orf72 DNA/RNA G4C2 G4

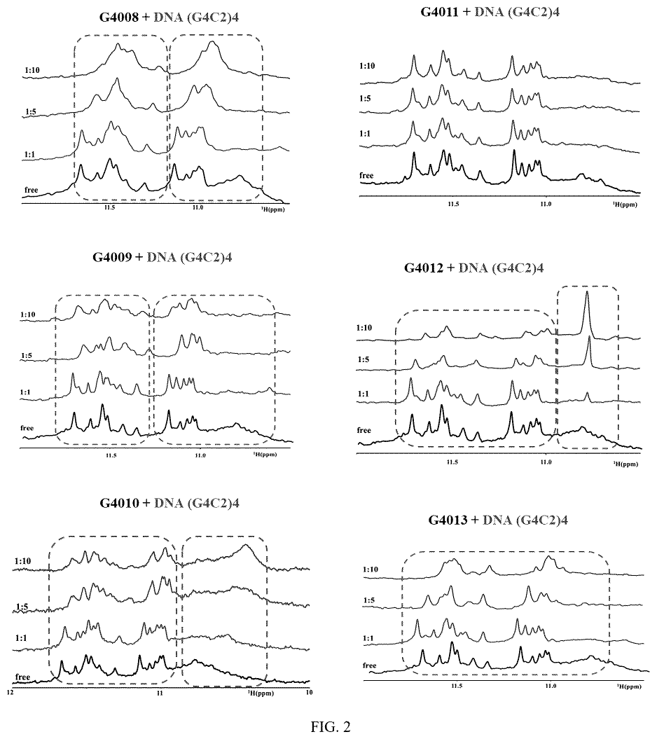

It is well known that C9orf72 DNA G4C2 repeats can fold into G4s including both antiparallel and parallel forms [13, 31]. We carried out NMR titration of these compounds with C9orf72 DNA/RNA G4C2 G4s. For C9orf72 DNA G4C2 sequence, the NMR titration was performed by using d(G4C2)₄. As shown in FIG. 2, compounds G4008, G4009, G4010, G4012 and G4013 show significant chemical shift changes in the imino proton region, indicating the binding of these compounds with C9orf72 d (G4C2)₄ (SEQ ID NO: 1).

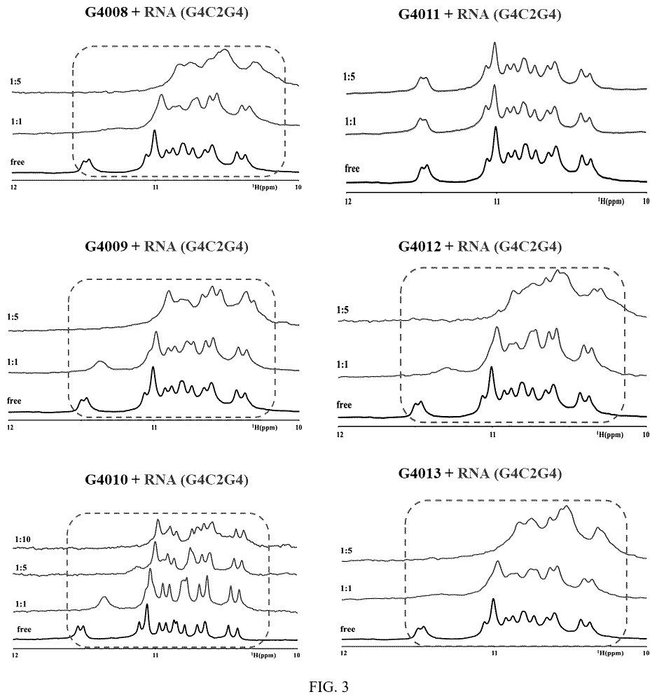

For C9orf72 RNA G4C2 sequence, the NMR titration was performed by using r(G4C2G4) (SEQ ID NO: 2), which can fold into a parallel G4 form [14]. As shown in FIG. 3, 5 out of 6 compounds, i.e., G4008, G4009, G4010, G4012, and G4013, show significant chemical shift changes in the imino proton region, indicating the binding of these compounds with C9orf72 r(G4C2G4). The further MTT assay of cellular toxicity shows that no relevant cytotoxicity was detected for G4011.

### Example 3—The Compounds can Recuse C9orf72 G4C2 Repeats-Related Pathologies at the cellular level

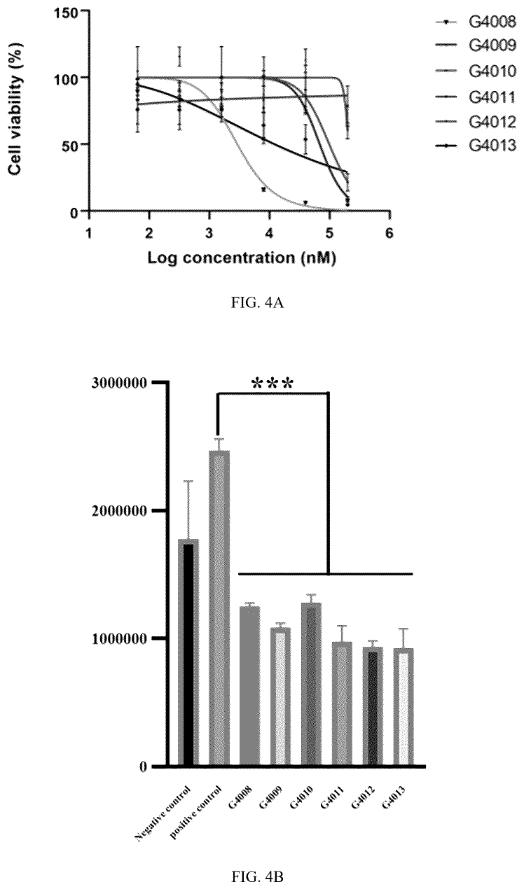

Cellular toxicity is critical to determine if cells could withstand and tolerate high concentrations of drug treatment without causing significant cell death. To evaluate the cytotoxicity of these 6 new compounds, MTT assay was applied to HT22 cells which is an immortalized mouse hippocampal neuronal cell line to measure cell viabilities (FIG. 4A). A high concentration of 200 μM was first applied and performed five times dilution onwards for a total of six data points (0.064 μM, 0.32 μM, 1.6 μM, 8 μM, 40 μM, 200 μM). The IC50 values calculated ranging from the highest toxicity to the lowest toxicity are as follow: G4008 (2.5 μM)>G4013 (10 μM)>G4009 (63 μM)>G4010 (79.4 μM) G4012 (199.5 μM)>>G4011 (no value). No relevant cytotoxicity was being detected for compound G4011 even up to 200 μM concentration and hence no inhibitory concentration was expected.

It has been reported that insufficient antioxidants were produced in C9-mutated oxidative stress and eventually neurodegeneration in ALS/FTD [32]. Therefore, it is essential to test whether the compounds have abilities to protect neuron cells from oxidative stress. Comparing the positive control (Rosup), where increased oxidative stress level was added, to the negative control, without drug treatment, all 6 compounds exhibited significant reduction in ROS level of cells induced by glutamate (FIG. 4B). Collectively, we chose G4008, G4009, G4010, G4012, and G4013 for further in vivo test in cell model expressing C9orf72 (G4C2)₂₉ sequence.

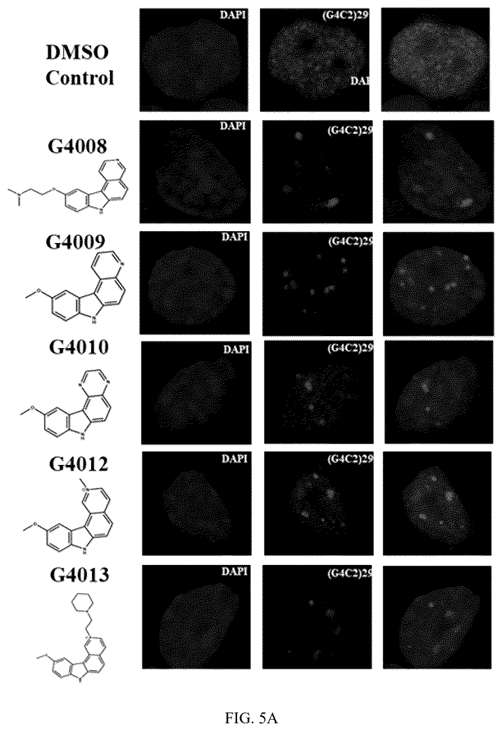

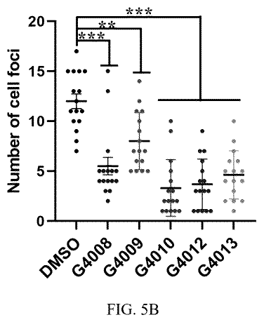

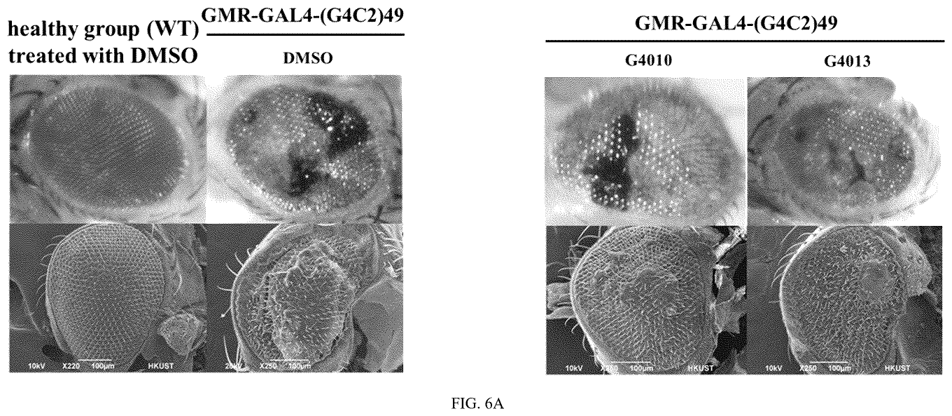

Since the NMR titration studies have unraveled the interactions between C9orf72 DNA/RNA G4C2 G4s and these compounds, the bioactivity of these compounds are hypothesized to work similarly. Hence, plasmid encoding the C9orf72 (G4C2)₂₉ DNA sequence (SEQ ID NO: 3) was transfected into Neuro2a cells used as an ALS/FTD cellular model. Expanded G4C2 repeats could result in ALS/FTD by sequestering RNA binding proteins (RBPs) and forming intranuclear RNA G4C2 foci which recruit specific RBPs such as hnRNP H. Based on the result of NMR titration in combination with the cell viability test (MTT assay), compounds G4008, G4009, G4010, G4012, and G4013 were selected for performing RNA FISH experiment to determine the bioactive effect on RNA G4C2 foci by immunostaining (FIG. 5A).

As shown in FIG. 5B, RNA foci number significantly reduced in cells treated overnight with G4008, G4010, G4012, and G4013. Considering the IC50 value in the MTT assay, we finally used G4010 and G4013 to perform bioactivity test on Drosophila fly model expressing C9orf72 (G4C2)₄₉ sequence.

### Example 4—The Compounds can Recuse C9orf72 G4C2 Repeats-Related Pathologies in Drosophila

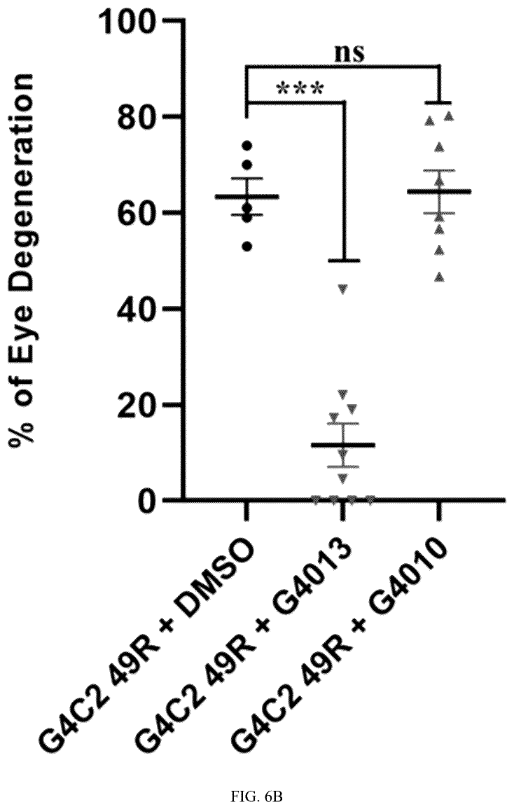

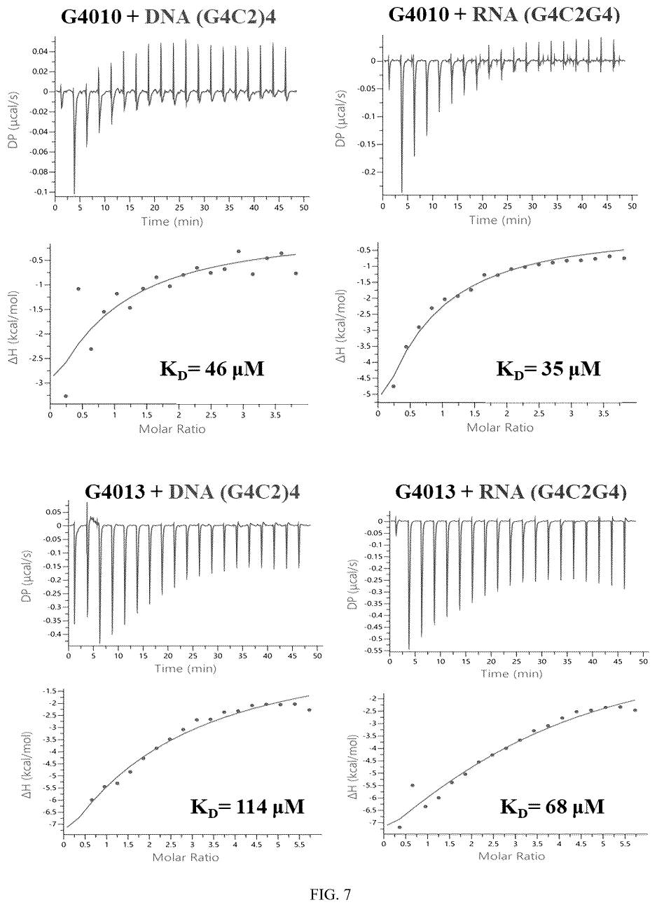

Furthermore, we tested the bioactive effects of G4010, G4012, and G4013 on C9orf72 ALS Drosophila. Therefore, GMR-GAL4-(G4C2)₄₉ Drosophila were engineered to express (G4C2)₄₉ RNA exclusively in eyes. The Drosophila models of C9-ALS that expressed pathogenic length 49 repeats, (G4C2)₄₉ confirmed that expanded G4C2 repeat sequences induce eye degeneration (FIG. 6B). The mutant and wild-type (WT) Drosophila were fed with compounds G4010 and G4013 in a working concentration of 100 μM in solid food at the larvae stage. In the external eye assay, we noted that the eyes of 7-day-old GMR-GAL4-(G4C2)₄₉ adult flies with dark spots were much rougher than age-matched WT flies, indicating eye degeneration. Intriguingly, G4013 treatment made eyes much smoother and more reflective (FIG. 6A), significantly suppressed eye degeneration in (G4C2)₄₉ flies, thus significantly rescuing the disorganized-structure eye phenotype. Altogether, these data show that G4013 rescues G4C2-repeats-caused eye defects in Drosophila (FIGS. 6A and 6B). However, G4010 cannot rescue the eye degeneration. Notably, the ITC experiments show that the binding affinity of G4010 and G4013 is ~100 fold stronger than chrexanthomycin A with K_{d}~46 and 114 μM with C9orf72 DNA (G4C2)₄ G4 and K_{d}~35 and 68 M with C9orf72 RNA (G4C2G4) G4, respectively (FIG. 7).

Therefore, this invention warrants these compounds' ability in binding specific targets of C9orf72 G4C2 expansions, which are the most common genetic cause of ALS and rescuing pathological signatures in ALS/FTD cell and Drosophila models. All these data suggest that G4013 can recognize C9orf72 G4C2 G4s with a high binding affinity and rescue G4C2-repeats-caused eye in Drosophila.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLARY EMBODIMENTS

Embodiment 1. A pharmaceutical composition, the composition comprising one or more compound selected from the group consisting of G4008, G4009, G4010, G4012, and G4013, or a pharmaceutically acceptable salt, solvate, or hydrate thereof and at least one pharmaceutically acceptable carrier and/or excipient.

Embodiment 2. A method of treating amyotrophic lateral sclerosis (ALS) and/or frontotemporal dementia (FTD) in a subject, the method comprising administering to the subject an effective amount of the composition of embodiment 1.

Embodiment 3. The method of embodiment 2, wherein the composition comprises G4008, G4010, G4012, or G4013.

Embodiment 4. The method of embodiment 2, wherein the composition comprises G4010 or G4013.

Embodiment 5. The method of embodiment 2, wherein the composition comprises compound G4013.

Embodiment 6. The method of embodiments 2-5, wherein the composition is administered 1, 2, 3, 4, or 5 times per week.

Embodiment 7. The method of embodiments 2-6, wherein the composition is administered at a dose of about 0.1 mg/kg to about 200 mg/kg.

Embodiment 8. The method of embodiments 2-7, wherein the composition is administered locally via intravitreal, intracranial, intradiscal, or systemically, via intramuscular, intravascular (e.g., intravenous), oral, intradermal, intranasal, intrathecal, or subcutaneous administration.

Embodiment 9. The method of embodiments 2-8, wherein the subject is a human.

Embodiment 10. The method of embodiments 2-9, wherein the one or more compound of the composition selectively targets and binds G-quadruplexes (G4s) formed by the GGGGCC (G4C2) hexanucleotide repeats (HREs) of C9orf72, (G4C2)₄.

Embodiment 11. The method of embodiments 2-10, wherein the administration of the composition ameliorates one or more symptoms of ASL and/or FTD by about 70%.

Embodiment 12. The method of embodiments 2-10, wherein the administration of the composition reduces the progression of ASL and/or FTD by about 70%.

Embodiment 13. The method of embodiment 3, wherein RNA foci number is significantly reduced by the administration of the composition comprising one or more compound selected from the group consisting of G4008, G4010, G4012, and G4013.

Embodiment 14. The method of embodiment 13, wherein the RNA foci number is reduced by about 50%.

Embodiment 15. The method of embodiments 2-14, wherein the administration of the composition reduces reactive oxygen species (ROS) level in the subject by about 40%.

Embodiment 16. The method of embodiment 5, wherein the administration of G4013 targets C9orf72 G4C2 G4s with a high binding affinity.

Embodiment 17. The method of embodiment 16, wherein the binding affinity (K_{d}) of G4013 with C9orf72 DNA (G4C2)₄ G4s is about 114 μM.

Embodiment 18. The method of embodiment 16, wherein the binding affinity (K_{d}) of G4013 with C9orf72 RNA (G4C2G4) G4s is about 68 μM.

Embodiment 19. The method of embodiment 2, wherein the composition comprises G4013 as only active agent.

### REFERENCES

[1] P. Masrori, P. Van Damme, Amyotrophic lateral sclerosis: a clinical review, Eur J Neurol, 27 (2020) 1918-1929.
[2] O. Hardiman, A. Al-Chalabi, A. Chio, E. M. Corr, G. Logroscino, W. Robberecht, P. J. Shaw, Z. Simmons, L. H. van den Berg, Amyotrophic lateral sclerosis, Nat Rev Dis Primers, 3 (2017) 17071.
[3] M. DeJesus-Hernandez, I. R. Mackenzie, B. F. Boeve, A. L. Boxer, M. Baker, N. J. Rutherford, A. M. Nicholson, N. A. Finch, H. Flynn, J. Adamson, N. Kouri, A. Wojtas, P. Sengdy, G. Y. Hsiung, A. Karydas, W. W. Seeley, K. A. Josephs, G. Coppola, D. H. Geschwind, Z. K. Wszolek, H. Feldman, D. S. Knopman, R. C. Petersen, B. L. Miller, D. W. Dickson, K. B. Boylan, N. R. Graff-Radford, R. Rademakers, Expanded GGGGCC hexanucleotide repeat in noncoding region of C9ORF72 causes chromosome 9p-linked FTD and ALS, Neuron, 72 (2011) 245-256.
[4] X. Liu, X. Zhao, J. He, S. Wang, X. Shen, Q. Liu, S. Wang, Advances in the Structure of GGGGCC Repeat RNA Sequence and Its Interaction with Small Molecules and Protein Partners, Molecules, 28 (2023).
[5] E. L. Feldman, S. A. Goutman, S. Petri, L. Mazzini, M. G. Savelieff, P. J. Shaw, G. Sobue, Amyotrophic lateral sclerosis, Lancet, 400 (2022) 1363-1380.
[6] C. M. Rodriguez, P. K. Todd, New pathologic mechanisms in nucleotide repeat expansion disorders, Neurobiol Dis, 130 (2019) 104515.
[7] Q. Zhu, J. Jiang, T. F. Gendron, M. McAlonis-Downes, L. Jiang, A. Taylor, S. Diaz Garcia, S. Ghosh Dastidar, M. J. Rodriguez, P. King, Y. Zhang, A. R. La Spada, H. Xu, L. Petrucelli, J. Ravits, S. Da Cruz, C. Lagier-Tourenne, D. W. Cleveland, Reduced C9ORF72 function exacerbates gain of toxicity from ALS/FTD-causing repeat expansion in C9orf72, Nat Neurosci, 23 (2020) 615-624.
[8] A. E. Renton, E. Majounie, A. Waite, J. Simón-Sánchez, S. Rollinson, J. R. Gibbs, J. C. Schymick, H. Laaksovirta, J. C. van Swieten, L. Myllykangas, H. Kalimo, A. Paetau, Y. Abramzon, A. M. Remes, A. Kaganovich, S. W. Scholz, J. Duckworth, J. H. Ding, D. W. Harmer, D. G. Hernandez, J. O. Johnson, K. Mok, M. Ryten, D. Trabzuni, R. J. Guerreiro, R. W. Orrell, J. Neal, A. Murray, J. Pearson, I. E. Jansen, D. Sondervan, H. Seelaar, D. Blake, K. Young, N. Halliwell, J. B. Callister, G. Toulson, A. Richardson, A. Gerhard, J. Snowden, D. Mann, D. Neary, M. A. Nalls, T. Peuralinna, L. Jansson, V. M. Isoviita, A. L. Kaivorinne, M. Hölttä-Vuori, E. Ikonen, R. Sulkava, M. Benatar, J. Wuu, A. Chiò, G. Restagno, G. Borghero, M. Sabatelli, D. Heckerman, E. Rogaeva, L. Zinman, J. D. Rothstein, M. Sendtner, C. Drepper, E. E. Eichler, C. Alkan, Z. Abdullaev, S. D. Pack, A. Dutra, E. Pak, J. Hardy, A. Singleton, N. M. Williams, P. Heutink, S. Pickering-Brown, H. R. Morris, P. J. Tienari, B. J. Traynor, I. Consortium, A Hexanucleotide Repeat Expansion in Is the Cause of Chromosome 9p21-Linked ALS-FTD, Neuron, 72 (2011) 257-268.
[9] A. Nordin, C. Akimoto, A. Wuolikainen, H. Alstermark, P. Jonsson, A. Birve, S. L. Marklund, K. S. Graffmo, K. Forsberg, T. Brannstrom, P. M. Andersen, Extensive size variability of the GGGGCC expansion in C9orf72 in both neuronal and non-neuronal tissues in 18 patients with ALS or FTD, Hum Mol Genet, 24 (2015) 3133-3142.
[10] I. Malik, C. P. Kelley, E. T. Wang, P. K. Todd, Molecular mechanisms underlying nucleotide repeat expansion disorders, Nat Rev Mol Cell Biol, 22 (2021) 589-607.
[11] R. Balendra, A. M. Isaacs, C9orf72-mediated ALS and FTD: multiple pathways to disease, Nat Rev Neurol, 14 (2018) 544-558.
[12] A. R. Haeusler, C. J. Donnelly, G. Periz, E. A. J. Simko, P. G. Shaw, M. S. Kim, N. J. Maragakis, J. C. Troncoso, A. Pandey, R. Sattler, J. D. Rothstein, J. Wang, nucleotide repeat structures initiate molecular cascades of disease, Nature, 507 (2014) 195−+.
[13] Y. Y. Geng, C. D. Liu, Q. X. Cai, Z. P. Luo, H. T. Miao, X. Shi, N,N. Xu, C. P. Fung, T. T. Choy, B. Yan, N. Li, P. Y. Qian, B. Zhou, G. Zhu, Crystal structure of parallel G-quadruplex formed by the two-repeat ALS- and FTD-related GGGGCC sequence, Nucleic Acids Res, 49 (2021) 5881-5890.
[14] Y. Y. Geng, C. D. Liu, N,N. Xu, M. C. Suen, H. T. Miao, Y. Y. Xie, B. C. Zhang, X. Q. Chen, Y. J. Song, Z. X. Wang, Q. X. Cai, G. Zhu, Crystal structure of a tetrameric RNA G-quadruplex formed by hexanucleotide repeat expansions of C9orf72 in ALS/FTD, Nucleic Acids Res, 52 (2024) 7961-7970.
[15] Z. F. Wang, A. Ursu, J. L. Childs-Disney, R. Guertler, W. Y. Yang, V. Bernat, S. G. Rzuczek, R. Fuerst, Y. J. Zhang, T. F. Gendron, I. Yildirim, B. G. Dwyer, J. E. Rice, L. Petrucelli, M. D. Disney, The Hairpin Form of r(G4C2)exp in c9ALS/FTD Is Repeat-Associated Non-ATG Translated and a Target for Bioactive Small Molecules, Cell Chem Biol, 26 (2019) 179-190e12.
[16] E. G. Conlon, L. Lu, A. Sharma, T. Yamazaki, T. Tang, N. A. Shneider, J. L. Manley, The C9ORF72 GGGGCC expansion forms RNA G-quadruplex inclusions and sequesters hnRNP H to disrupt splicing in ALS brains, Elife, 5 (2016).
[17] R. J. Mead, N. Shan, H. J. Reiser, F. Marshall, P. J. Shaw, Amyotrophic lateral sclerosis: a neurodegenerative disorder poised for successful therapeutic translation, Nat Rev Drug Discov, 22 (2023) 185-212.
[18] U. Ansari, M. Alam, D. Nadora, Z. Muttalib, V. Chen, I. Taguinod, M. FitzPatrick, J. Wen, Z. Ansari, F. Lui, Assessing the efficacy of amyotrophic lateral sclerosis drugs in slowing disease progression: A literature review, AIMS Neurosci, 11 (2024) 166-177.
[19] S. A. Johnson, T. Fang, F. De Marchi, D. Neel, D. Van Weehaeghe, J. D. Berry, S. Paganoni, Pharmacotherapy for Amyotrophic Lateral Sclerosis: A Review of Approved and Upcoming Agents, Drugs, 82 (2022) 1367-1388.
[20] H. A. Blair, Tofersen: First Approval, Drugs, 83 (2023) 1039-1043.
[21] D. B. Tiz, L. Bagnoli, O. Rosati, F. Marini, C. Santi, L. Sancineto, FDA-Approved Small Molecules in 2022: Clinical Uses and Their Synthesis, Pharmaceutics, 14 (2022).
[22] P. Neupane, P. K. Thada, P. Singh, A. R. Faisal, N. Rai, P. Poudel, M. S. Waleed, J. Quinonez, S. Ruxmohan, E. Jain, Investigating Edaravone Use for Management of Amyotrophic Lateral Sclerosis (ALS): A Narrative Review, Cureus, 15 (2023) e33746.
[23] M. C. Bellingham, A Review of the Neural Mechanisms of Action and Clinical Efficiency of Riluzole in Treating Amyotrophic Lateral Sclerosis: What have we Learned in the Last Decade?, Cns Neurosci Ther, 17 (2011) 4-31.
[24] B. R. Brooks, P. Bettica, S. Cazzaniga, Riluzole Oral Suspension: Bioavailability Following Percutaneous Gastrostomy Tube-modeled Administration Versus Direct Oral Administration, Clin Ther, 41 (2019) 2490-2499.
[25] J. Wymer, S. Apple, A. Harrison, B. A. Hill, Pharmacokinetics, Bioavailability, and Swallowing Safety With Riluzole Oral Film, Clin Pharmacol Drug Dev, 12 (2023) 57-64.
[26] Y. Sun, M. Benatar, J. Mascias Cadavid, D. Ennist, P. Wicks, K. Staats, M. Beauchamp, S. Jhooty, G. Pattee, A. Brown, T. Bertorini, P. Barkhaus, M. Bromberg, G. Carter, R. Bedlack, X. Li, ALSUntangled #71: Nuedexta, Amyotroph Lateral Scler Frontotemporal Degener, 25 (2024) 218-222.
[27] van Meerloo J, Kaspers G J, Cloos J. Cell sensitivity assays: the MTT assay. Methods Mol Biol, 2011; 731:237-45. doi: 10.1007/978-1-61779-080-5_20. PMID: 21516412.
[28] Z. Su, Y. Zhang, T. F. Gendron, P. O. Bauer, J. Chew, W. Y. Yang, E. Fostvedt, K. Jansen-West, V. V. Belzil, P. Desaro, A. Johnston, K. Overstreet, S. Y. Oh, P. K. Todd, J. D. Berry, M. E. Cudkowicz, B. F. Boeve, D. Dickson, M. K. Floeter, B. J. Traynor, C. Morelli, A. Ratti, V. Silani, R. Rademakers, R. H. Brown, J. D. Rothstein, K. B. Boylan, L. Petrucelli, M. D. Disney, Discovery of a Biomarker and Lead Small Molecules to Target r(GGGGCC)-Associated Defects in c9FTD/ALS, Neuron, 84 (2014) 239.
[29] R. Simone, P. Fratta, S. Neidle, G. N. Parkinson, A. M. Isaacs, G-quadruplexes: Emerging roles in neurodegenerative diseases and the non-coding transcriptome, FEBS Lett, 589 (2015) 1653-1668.
[30] A. Cheng, C. Liu, W. Ye, D. Huang, W. She, X. Liu, C. P. Fung, N. Xu, M. C. Suen, W. Ye, H. H. Y. Sung, I. D. Williams, G. Zhu, P. Y. Qian, Selective C9orf72 G-Quadruplex-Binding Small Molecules Ameliorate Pathological Signatures of ALS/FTD Models, J Med Chem, 65 (2022) 12825-12837.
[31] B. Zhou, C. Liu, Y. Geng, G. Zhu, Topology of a G-quadruplex DNA formed by C9orf72 hexanucleotide repeats associated with ALS and FTD, Sci Rep, 5 (2015) 16673.
[32] A. Birger, I. Ben-Dor, M. Ottolenghi, T. Turetsky, Y. Gil, S. Sweetat, L. Perez, V. Belzer, N. Casden, D. Steiner, M. Izrael, E. Galun, E. Feldman, O. Behar, B. Reubinoff, Human iPSC-derived astrocytes from ALS patients with mutated C9ORF72 show increased oxidative stress and neurotoxicity, EBioMedicine, 50 (2019) 274-289.

We claim:

1. A pharmaceutical composition, the composition comprising one or more compound selected from the group consisting of G4008, G4009, G4010, G4012, and G4013, or a pharmaceutically acceptable salt, solvate, or hydrate thereof and at least one pharmaceutically acceptable carrier and/or excipient.

2. A method of treating amyotrophic lateral sclerosis (ALS) and/or frontotemporal dementia (FTD) in a subject, the method comprising administering to the subject an effective amount of the composition of claim 1.

3. The method of claim 2, wherein the composition comprises G4008, G4010, G4012, or G4013.

4. The method of claim 2, wherein the composition comprises G4010 or G4013.

5. The method of claim 2, wherein the composition comprises compound G4013.

6. The method of claim 2, wherein the composition is administered 1, 2, 3, 4, or 5 times per week.

7. The method of claim 2, wherein the composition is administered at a dose of about 0.1 mg/kg to about 200 mg/kg.

8. The method of claim 2, wherein the composition is administered locally via intravitreal, intracranial, intradiscal, or systemically, via intramuscular, intravascular (e.g., intravenous), oral, intradermal, intranasal, intrathecal, or subcutaneous administration.

9. The method of claim 2, wherein the subject is a human.

10. The method of claim 2, wherein the one or more compound of the composition selectively targets and binds G-quadruplexes (G4s) formed by the GGGGCC (G4C2) hexanucleotide repeats (HREs) of C9orf72, (G4C2)₄.

11. The method of claim 2, wherein the administration of the composition ameliorates one or more symptoms of ASL and/or FTD by about 70%.

12. The method of claim 2, wherein the administration of the composition reduces the progression of ASL and/or FTD by about 70%.

13. The method of claim 3, wherein RNA foci number is significantly reduced by the administration of the composition comprising one or more compound selected from the group consisting of G4008, G4010, G4012, and G4013.

14. The method of claim 13, wherein the RNA foci number is reduced by about 50%.

15. The method of claim 2, wherein the administration of the composition reduces reactive oxygen species (ROS) level in the subject by about 40%.

16. The method of claim 2, wherein the administration of G4013 targets C9orf72 G4C2 G4s with a high binding affinity.

17. The method of claim 16, wherein the binding affinity (K_{d}) of G4013 with C9orf72 DNA (G4C2)₄ G4s is about 114 μM.

18. The method of claim 16, wherein the binding affinity (K_{d}) of G4013 with C9orf72 RNA (G4C2G4) G4s is about 68 μM.

19. The method of claim 2, wherein the composition comprises G4013 as only active agent.
